(19) United States
(12) Patent Application Publication
(10) Pub. No.: US 2016/0058977 A1
(43) Pub. Date: Mar. 3, 2016

(54) INTRAVASCULAR DEVICES, SYSTEMS, AND METHODS HAVING AN ADHESIVE FILLED DISTAL TIP ELEMENT

(71) Applicant: Volcano Corporation, San Diego, CA (US)

(72) Inventors: David H. Burkett, Temecula, CA (US); Chris Szunyog, Murrieta, CA (US)

(21) Appl. No.: 14/837,519

(22) Filed: Aug. 27, 2015

Related U.S. Application Data
(60) Provisional application No. 62/042,971, filed on Aug. 28, 2014.

(51) Int. Cl. A61M 25/09; A61B 5/0215; A61B 5/026; A61B 5/00

(52) U.S. Cl. CPC A61M 25/09; A61B 5/6851; A61B 5/0215; A61B 5/026; A61M 2025/0002

(57) ABSTRACT

Intravascular devices, systems, and methods are disclosed. In some instances, the intravascular device is a guide wire with an adhesive filled distal coil. For example, in some implementations a sensing guide wire includes a flexible elongate member; a sensing element coupled to a distal portion of the flexible elongate member; and a flexible element filled with a flexible adhesive extending distally from the sensing element. Methods of making, manufacturing, and/or assembling such intravascular devices and associated systems are also provided.

### CROSS REFERENCE TO RELATED APPLICATIONS

The present application claims priority to and the benefit of the U.S. Provisional Patent Application Nos. 62/042,971, filed Aug. 28, 2014, which is hereby incorporated by reference in its entirety.

### TECHNICAL FIELD

The present disclosure relates to intravascular devices, systems, and methods. In some embodiments, the intravascular devices are guide wires that include a distal coil filled with a flexible adhesive.

### BACKGROUND

Heart disease is very serious and often requires emergency operations to save lives. A main cause of heart disease is the accumulation of plaque inside the blood vessels, which eventually occludes the blood vessels. Common treatment options available to open up the occluded vessel include balloon angioplasty, rotational atherectomy, and intravascular stents. Traditionally, surgeons have relied on X-ray fluoroscopic images that are planar images showing the external shape of the silhouette of the lumen of blood vessels to guide treatment. Unfortunately, with X-ray fluoroscopic images, there is a great deal of uncertainty about the exact extent and orientation of the stenosis responsible for the occlusion, making it difficult to find the exact location of the stenosis. In addition, though it is known that restenosis can occur at the same place, it is difficult to check the condition inside the vessels after surgery with X-ray.

A currently accepted technique for assessing the severity of a stenosis in a blood vessel, including ischemia causing lesions, is fractional flow reserve (FFR). FFR is a calculation of the ratio of a distal pressure measurement (taken on the distal side of the stenosis) relative to a proximal pressure measurement (taken on the proximal side of the stenosis). FFR provides an index of stenosis severity that allows determination as to whether the blockage limits blood flow within the vessel to an extent that treatment is required. The normal value of FFR in a healthy vessel is 1.00, while values less than about 0.80 are generally deemed significant and require treatment.

Often intravascular catheters and guide wires are utilized to measure the pressure within the blood vessel, visualize the inner lumen of the blood vessel, and/or otherwise obtain data related to the blood vessel. To date, guide wires containing pressure sensors, imaging elements, and/or other electronic, optical, or electro-optical components have suffered from reduced performance characteristics compared to standard guide wires that do not contain such components. For example, the handling performance of previous guide wires containing electronic components have been hampered, in some instances, by the limited space available for the core wire after accounting for the space needed for the conductors or communication lines of the electronic component(s), the stiffness of the rigid housing containing the electronic component(s), and/or other limitations associated with providing the functionality of the electronic components in the limited space available within a guide wire.

Further, a problem with existing pressure and flow guide wires is that the coil(s) defining the distal tip of the device can be fragile and prone to unwanted bending or kinking. In that regard, the small diameter and high flexibility of the coil(s) limits the structural integrity that can be provided. Further, the rigid nature of the sensor housing adjacent to the coil(s) causes additional stress to be applied to the coil(s) during use, especially when traversing complex vasculature with many curves and turns. As a result, the handling and performance of the guide wires can be reduced because of the limitations of the coil(s).

Accordingly, there remains a need for improved intravascular devices, systems, and methods that include one or more electronic, optical, or electro-optical components.

### SUMMARY

The present disclosure is directed to intravascular devices, systems, and methods that include a guide wire having a distal coil filled with a flexible adhesive.

In some instances, a sensing guide wire is provided that includes: a flexible elongate member; a sensing element coupled to a distal portion of the flexible elongate member; and a flexible element extending distally from the sensing element, wherein the flexible element is at least partially filled along a longitudinal axis of the flexible element with one or more flexible adhesives. The sensing element includes at least one of a pressure sensor and a flow sensor in some implementations. A core element can be positioned within the flexible element. In that regard, the core element can be coupled to a shaping ribbon such that the core element extends only a portion of a total length of the flexible element. The flexible element can include a coil and the one or more flexible adhesives can include an adhesive selected from the group of adhesives consisting of urethane adhesives and silicone adhesives. In some instances, two different adhesives are utilized, where one of the adhesives has an increased flexibility relative to the other. In that regard, the more flexible adhesive is positioned distal of the other adhesive within a central lumen of the flexible element in some instances. The flexible adhesive can be spaced from the outer surface of the coil by a distance of at least ten percent of a diameter of a wire material forming the coil. In some instances, the coil has an outer diameter of approximately 0.014″, 0.018″, or 0.035″.

In some instances, a method of forming a sensing guide wire is provided that includes: coupling a sensing element to a distal portion of a flexible elongate member; coupling a flexible element to the distal portion of the flexible elongate member such that the flexible element extends distally from the sensing element; and at least partially filling the flexible element with one or more flexible adhesives.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic, schematic side view of an intravascular device according to an embodiment of the present disclosure.

FIG. 2 is a diagrammatic, schematic side view of a distal portion of the intravascular device of FIG. 1 according to an embodiment of the present disclosure.

FIG. 3 is a cross-sectional side view of the distal portion of the intravascular device of FIGS. 1 and 2 taken along section line 3-3 of FIG. 2 according to an embodiment of the present disclosure.

FIG. 4 is a magnified cross-sectional side view of a section of the distal portion of the intravascular device of FIGS. 1-3 according to an embodiment of the present disclosure.

FIG. 5 is a magnified cross-sectional side view of a section of the distal portion of an intravascular device according to another embodiment of the present disclosure.

FIG. 6 is a magnified cross-sectional side view of a section of the distal portion of an intravascular device according to another embodiment of the present disclosure.

FIG. 7 is a magnified cross-sectional side view of a section of the distal portion of an intravascular device according to another embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As used herein, “flexible elongate member” or “elongate flexible member” includes at least any thin, long, flexible structure that can be inserted into the vasculature of a patient. While the illustrated embodiments of the “flexible elongate members” of the present disclosure have a cylindrical profile with a circular cross-sectional profile that defines an outer diameter of the flexible elongate member, in other instances all or a portion of the flexible elongate members may have other geometric cross-sectional profiles (e.g., oval, rectangular, square, elliptical, etc.) or non-geometric cross-sectional profiles. Flexible elongate members include, for example, guide wires and catheters. In that regard, catheters may or may not include a lumen extending along its length for receiving and/or guiding other instruments. If the catheter includes a lumen, the lumen may be centered or offset with respect to the cross-sectional profile of the device.

In most embodiments, the flexible elongate members of the present disclosure include one or more electronic, optical, or electro-optical components. For example, without limitation, a flexible elongate member may include one or more of the following types of components: a pressure sensor, a flow sensor, a temperature sensor, an imaging element, an optical fiber, an ultrasound transducer, a reflector, a mirror, a prism, an ablation element, an RF electrode, a conductor, and/or combinations thereof. Generally, these components are configured to obtain data related to a vessel or other portion of the anatomy in which the flexible elongate member is disposed. Often the components are also configured to communicate the data to an external device for processing and/or display. In some aspects, embodiments of the present disclosure include imaging devices for imaging within the lumen of a vessel, including both medical and non-medical applications. However, some embodiments of the present disclosure are particularly suited for use in the context of human vasculature. Imaging of the intravascular space, particularly the interior walls of human vasculature can be accomplished by a number of different techniques, including ultrasound (often referred to as intravascular ultrasound (“IVUS”) and intracardiac echocardiography (“ICE”)) and optical coherence tomography (“OCT”). In other instances, infrared, thermal, or other imaging modalities are utilized.

The electronic, optical, and/or electro-optical components of the present disclosure are often disposed within a distal portion of the flexible elongate member. As used herein, “distal portion” of the flexible elongate member includes any portion of the flexible elongate member from the mid-point to the distal tip. As flexible elongate members can be solid, some embodiments of the present disclosure will include a housing portion at the distal portion for receiving the electronic components. Such housing portions can be tubular structures attached to the distal portion of the elongate member. Some flexible elongate members are tubular and have one or more lumens in which the electronic components can be positioned within the distal portion.

The electronic, optical, and/or electro-optical components and the associated communication lines are sized and shaped to allow for the diameter of the flexible elongate member to be very small. For example, the outside diameter of the elongate member, such as a guide wire or catheter, containing one or more electronic, optical, and/or electro-optical components as described herein are between about 0.0007″ (0.0178 mm) and about 0.118″ (3.0 mm), with some particular embodiments having outer diameters of approximately 0.014″ (0.3556 mm), approximately 0.018″ (0.4572 mm), and approximately 0.035″ (0.889 mm). As such, the flexible elongate members incorporating the electronic, optical, and/or electro-optical component(s) of the present application are suitable for use in a wide variety of lumens within a human patient besides those that are part or immediately surround the heart, including veins and arteries of the extremities, renal arteries, blood vessels in and around the brain, and other lumens.

“Connected” and variations thereof as used herein includes direct connections, such as being glued or otherwise fastened directly to, on, within, etc. another element, as well as indirect connections where one or more elements are disposed between the connected elements.

“Secured” and variations thereof as used herein includes methods by which an element is directly secured to another element, such as being glued or otherwise fastened directly to, on, within, etc. another element, as well as indirect techniques of securing two elements together where one or more elements are disposed between the secured elements.

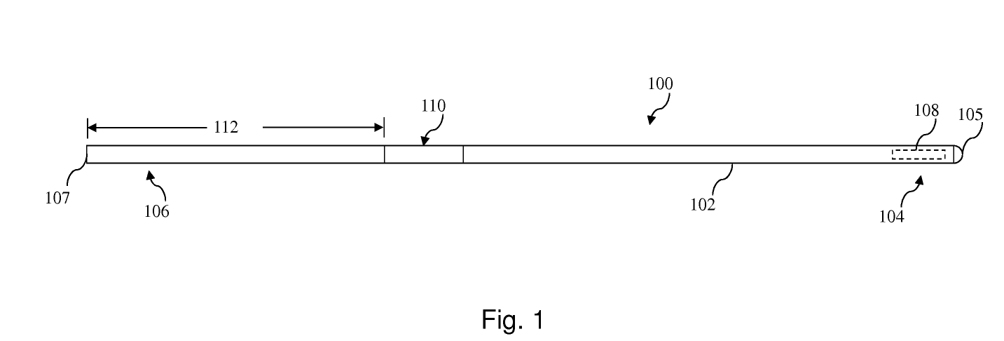

Referring now to FIG. 1, shown therein is a portion of an intravascular device 100 according to an embodiment of the present disclosure. In that regard, the intravascular device 100 includes a flexible elongate member 102 having a distal portion 104 adjacent a distal tip 105 and a proximal portion 106 adjacent a proximal end 107. A component 108 is positioned within the distal portion 104 of the flexible elongate member 102 proximal of the distal tip 105. Generally, the component 108 is representative of one or more electronic, optical, or electro-optical components. In that regard, the component 108 is a pressure sensor, a flow sensor, a temperature sensor, an imaging element, an optical fiber, an ultrasound transducer, a reflector, a mirror, a prism, an ablation element, an RF electrode, a conductor, and/or combinations thereof. The specific type of component or combination of components can be selected based on an intended use of the intravascular device. In some instances, the component 108 is positioned less than 10 cm, less than 5, or less than 3 cm from the distal tip 105. In some instances, the component 108 is positioned within a housing of the flexible elongate member 102. In that regard, the housing is a separate component secured to the flexible elongate member 102 in some instances. In other instances, the housing is integrally formed as a part of the flexible elongate member 102.

The intravascular device 100 also includes a connector 110 adjacent the proximal portion 106 of the device. In that regard, the connector 110 is spaced from the proximal end 107 of the flexible elongate member 102 by a distance 112. Generally, the distance 112 is between 0% and 50% of the total length of the flexible elongate member 102. While the total length of the flexible elongate member can be any length, in some embodiments the total length is between about 1300 mm and about 4000 mm, with some specific embodiments have a length of 1400 mm, 1900 mm, and 3000 mm. Accordingly, in some instances the connector 110 is positioned at the proximal end 107. In other instances, the connector 110 is spaced from the proximal end 107. For example, in some instances the connector 110 is spaced from the proximal end 107 between about 0 mm and about 1400 mm. In some specific embodiments, the connector 110 is spaced from the proximal end by a distance of 0 mm, 300 mm, and 1400 mm.

The connector 110 is configured to facilitate communication between the intravascular device 100 and another device. More specifically, in some embodiments the connector 110 is configured to facilitate communication of data obtained by the component 108 to another device, such as a computing device or processor. Accordingly, in some embodiments the connector 110 is an electrical connector. In such instances, the connector 110 provides an electrical connection to one or more electrical conductors that extend along the length of the flexible elongate member 102 and are electrically coupled to the component 108. In some embodiments the electrical conductors are embedded within a core of the flexible elongate member. In other embodiments, the connector 110 is an optical connector. In such instances, the connector 110 provides an optical connection to one or more optical communication pathways (e.g., fiber optic cable) that extend along the length of the flexible elongate member 102 and are optically coupled to the component 108. Similarly, in some embodiments the optical fibers are embedded within a core of the flexible elongate member. Further, in some embodiments the connector 110 provides both electrical and optical connections to both electrical conductor(s) and optical communication pathway(s) coupled to the component 108. In that regard, it should be noted that component 108 is comprised of a plurality of elements in some instances. The connector 110 is configured to provide a physical connection to another device, either directly or indirectly. In some instances, the connector 110 is configured to facilitate wireless communication between the intravascular device 100 and another device. Generally, any current or future developed wireless protocol(s) may be utilized. In yet other instances, the connector 110 facilitates both physical and wireless connection to another device.

As noted above, in some instances the connector 110 provides a connection between the component 108 of the intravascular device 100 and an external device. Accordingly, in some embodiments one or more electrical conductors, one or more optical pathways, and/or combinations thereof extend along the length of the flexible elongate member 102 between the connector 110 and the component 108 to facilitate communication between the connector 110 and the component 108. In some instances, at least one of the electrical conductors and/or optical pathways is embedded within the core of the flexible elongate member 102, as described in U.S. Provisional Patent Application No. 61/935,113, filed Feb. 3, 2014, published as U.S. Patent Application Publication No. 2015/0217090 on Aug. 6, 2015, which is hereby incorporated by reference in its entirety. Generally, any number of electrical conductors, optical pathways, and/or combinations thereof can extend along the length of the flexible elongate member 102 between the connector 110 and the component 108, embedded in the core or not. In some instances, between one and ten electrical conductors and/or optical pathways extend along the length of the flexible elongate member 102 between the connector 110 and the component 108. The number of communication pathways and the number of electrical conductors and optical pathways extending along the length of the flexible elongate member 102 is determined by the desired functionality of the component 108 and the corresponding elements that define component 108 to provide such functionality.

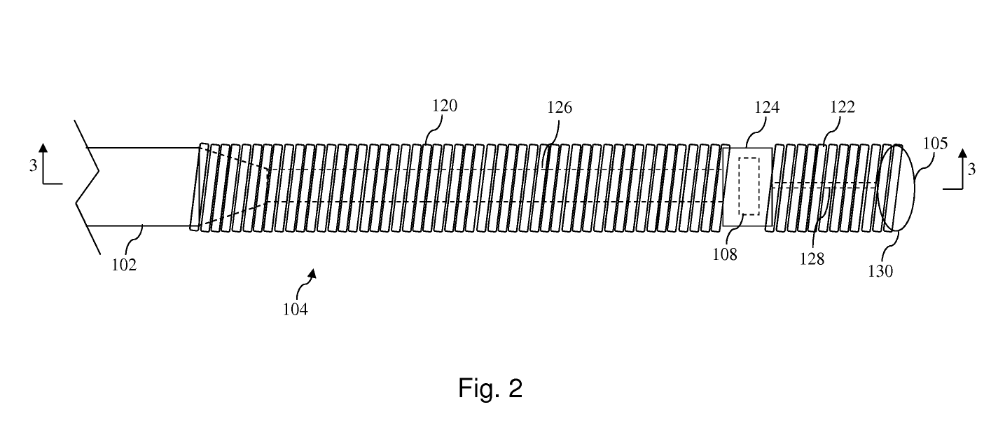

Referring now to FIGS. 2-4, shown therein are aspects of the intravascular devices of the present disclosure that include a coil filled with a flexible adhesive. In that regard, one of the major issues associated with existing functional guide wires is poor mechanical performance as compared to frontline guide wires. The use of an adhesive filled coil at the distal tip of the intravascular device in accordance with the present disclosure has been found to significantly improve the mechanical performance of the guide wires, including the durability of the distal coil.

Referring now to FIG. 2, shown therein is a diagrammatic, schematic side view of the distal portion 104 of the intravascular device 100 according to an embodiment of the present disclosure. As shown, the distal portion 104 includes a proximal flexible element 120 and a distal flexible element 122 on each side of a housing 124 containing component 108. A core member 126 extends through the proximal flexible element 120. Similarly, a core member 128 extends through the distal flexible element 122. Generally, the core members 126, 128 are sized, shaped, and/or formed out of particular material(s) to create a desired mechanical performance for the distal portion 104 of the intravascular device 100. In that regard, in some instances the core member 128 is coupled to a shaping ribbon. For example, in some particular implementations the core member 128 is coupled to a shaping ribbon utilizing a multi-flat transition as described in U.S. Patent Application No. 62/027,556 filed Jul. 22, 2014 (Attorney Docket No. 44755.1466/FM-0114), which is hereby incorporated by reference in its entirety.

The proximal and distal flexible elements 120, 122 can be any suitable flexible element, including coils, polymer tubes, and/or coil-embedded polymer tubes. In the illustrated embodiment the proximal flexible element 120 and the distal flexible element 122 are coils. As discussed in greater detail below, the distal flexible element 122 is filled, or partially filled, with one or more flexible adhesives to improve the mechanical performance and durability of the intravascular device 100. Further, a solder ball 130 or other suitable element is secured to the distal end of the distal flexible element 122. As shown, the solder ball 130 defines the distal tip 105 of the intravascular device 100 with an atraumatic tip suitable for advancement through patient vessels, such as vasculature. In some embodiments, a flow sensor is positioned at the distal tip 105 instead of the solder ball 130.

The distal portion 104 of the intravascular device 100—as well as the proximal portion 106 and the flexible elongate member 102—may be formed using any suitable approach so long as the distal flexible element 122 is filled with a flexible adhesive in accordance with the present disclosure. Accordingly, in some implementations the intravascular device 100 includes features similar to the distal, intermediate, and/or proximal sections described in one or more of U.S. Pat. No. 5,125,137, U.S. Pat. No. 5,873,835, U.S. Pat. No. 6,106,476, U.S. Pat. No. 6,551,250, U.S. patent application Ser. No. 13/931,052, filed Jun. 28, 2013, published as U.S. Patent Application Publication No. 2014/0005543 on Jan. 2, 2014, U.S. patent application Ser. No. 14/135,117, filed Dec. 19, 2013, published as U.S. Patent Application Publication No. 2014/0180141 on Jun. 26, 2014, U.S. patent application Ser. No. 14/137,364, filed Dec. 20, 2013, published as U.S. Patent Application Publication No. 2014/0187980 on Jul. 3, 2014, U.S. patent application Ser. No. 14/139,543, filed Dec. 23, 2013, published as U.S. Patent Application Publication No. 2014/0187984 on Jul. 3, 2014, U.S. patent application Ser. No. 14/143,304, filed Dec. 30, 2013, published as U.S. Patent Application Publication No. 2014/0187874 on Jul. 3, 2014, and U.S. Provisional Patent Application No. 61/935,113, filed Feb. 3, 2014, published as U.S. Patent Application Publication No. 2015/0217090 on Aug. 6, 2015, each of which is hereby incorporated by reference in its entirety.

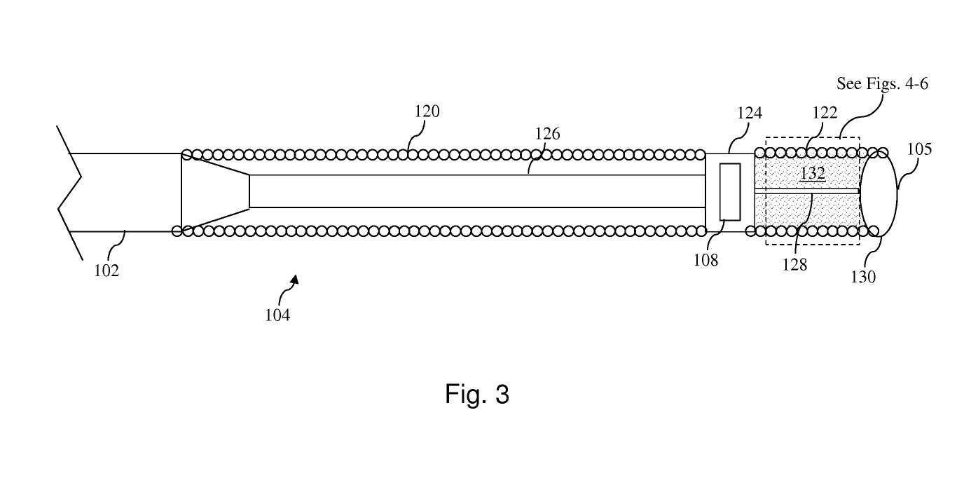

Referring now to FIG. 3, shown therein is a cross-sectional side view of the distal portion 104 of the intravascular device 100 taken along section line 3-3 of FIG. 2 according to an embodiment of the present disclosure. As shown, the distal flexible element 122 is filled, or partially filled, with one or more flexible materials 132. The material(s) 132 is(are) configured to improve the mechanical integrity of the distal flexible element 122, while maintaining sufficient flexibility for use of the intravascular device in tortuous vessels. In some instances, the material 132 includes one or more flexible adhesives such as Dymax 1901-M, Dymax 9001, Loctite 5248, etc. In that regard, in some implementations the flexible adhesives have a minimum durometer of shore hardness 25A to a maximum durometer of shore hardness 60D. In the context of a coil distal flexible element 122, the flexible adhesive can secure the windings in place relative to one another, which helps protect the distal portion 104 of the intravascular device 100 from damage during subsequent manufacturing steps, transport, and/or use. In that regard, the adhesive(s) will lock the coil position relative to itself and the distal core 128. This can greatly minimize potential for damage to the tip due to stretching of the coils during handling or use. All tip coils need to have some initial stretch because a stacked coil would have significantly high column strength and could overlap coils when put into tortuosity. However, the more stretch in the coil, the more easily the coil can be damaged. Embodiments of the present disclosure help prevent this from happening.

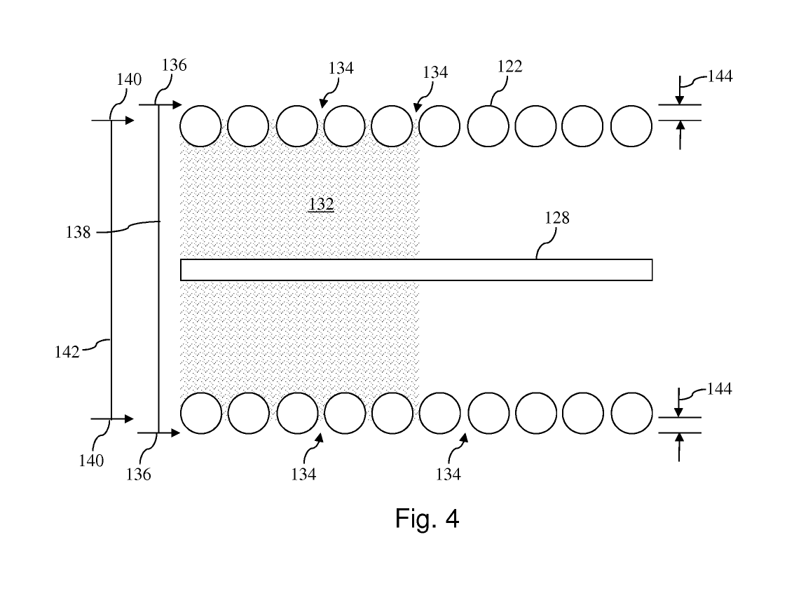

Referring now to FIGS. 4-6, shown therein are magnified cross-sectional side views of the distal portion 104 of the intravascular device 100 according to various exemplary embodiments of the present disclosure. Referring initially to FIG. 4, the material 132 partially fills a central lumen of the distal flexible element 122. As shown, the material 132 at least partially fills spaces 134 between adjacent windings of the distal flexible element 122. In that regard, in some instances the material 132 is introduced into the central lumen of the distal flexible element 122 through the spaces 134 (e.g., by wicking, injecting, flowing, and/or combinations thereof). In some instances, the material 132 is introduced into the central lumen of the distal flexible element 122 through an opening in one of the ends of the flexible element 122 and filled until the material at least partially fills the spaces 134. In that regard, the material 132 is spaced from the outer most surface(s) 136 of the distal flexible element 122 in some embodiments.

As shown, the outer most surface(s) 136 of the distal flexible element 122 has (have) a diameter 138. Generally, the diameter 138 is approximately equal to the maximum desired outer diameter of the intravascular device 100. Accordingly, in some particular implementations the diameter 138 is about 0.014″, 0.018″, or 0.035″. The outer boundary 140 of the material 132 has a diameter 142 that is smaller than the diameter 138 of the distal flexible element 122 such that the material is spaced from the outer most surface(s) 136 of the distal flexible element. In some instances, the diameter 142 is less than the diameter 138 by between about 0.0001″ and about 0.005″, between about 0.005″ and about 0.001″, or other suitable range. Accordingly, in some instances, the diameter 142 is about 0.013″, 0.017″, or 0.034″. In some implementations the diameter 142 is equal to the diameter 138 of the distal flexible element or reduced by up to two times the diameter of the tip coil wire utilized to form the coil. Accordingly, for a 0.014″ outer diameter tip coil using 0.0025″ diameter wire material, the diameter 142 may range from 0.009″ to 0.014″. Similarly, in some implementations, the diameter 142 is less than the diameter 138 by a percentage of the diameter of the wire material used to form the coil, such as ten percent, twenty percent, twenty-five percent, fifty percent or more the wire diameter.

By spacing the material 132 from the outer most surface(s) 136 of the distal flexible element 122, the tactile response to a user associated with the distal flexible element 122 contacting anatomical structures is maintained. On the other hand, if the material 132 completely covers the outer surface(s) of the distal flexible element 122, then a continuous surface of material 132 may be formed that can affect the tactile response of the intravascular device 100 when in use.

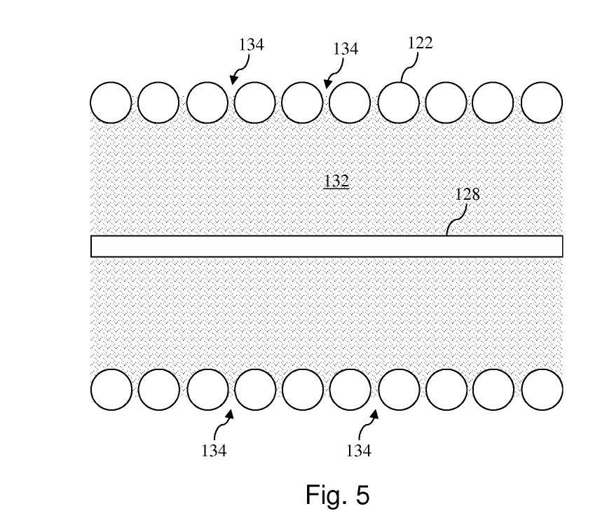

In the embodiment of FIG. 4, the material 132 extends along only a portion of the length of the distal flexible element 122. In particular, the material 132 is positioned only within a proximal section of the distal flexible element 122 such that a distal section of the distal flexible element 122 does not include the material 132. In that regard, the material 132 extends along the distal flexible element 122 between about 1 percent and about 100 percent of the length of the distal flexible element 122. In some instances, the distal flexible element 122 has a length of approximately 3 cm and the material 132 extends from a proximal end of the distal flexible element a distance between about 1 mm and about 20 mm. FIG. 5 illustrates another embodiment where the material 132 substantially fills the entire central lumen of the distal flexible element 122.

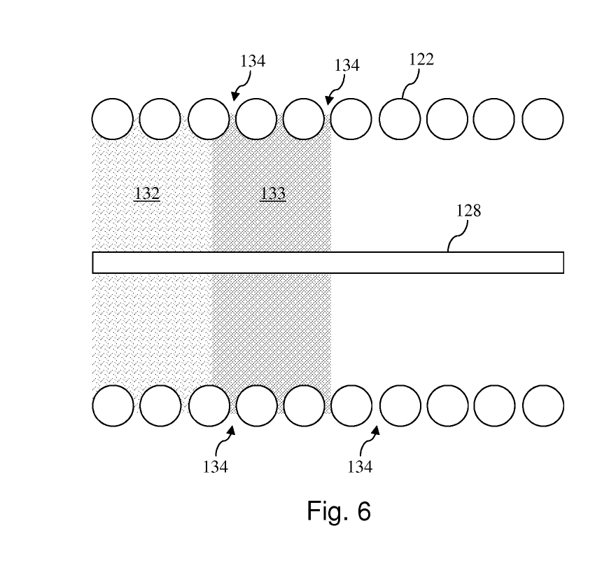

Referring now to FIG. 6, shown therein is another embodiment where multiple flexible materials are utilized within the distal flexible element 122. In particular, the material 132 fills a portion of the distal flexible element 122 and another material 133 fills another portion of the distal flexible element 122. In the illustrated embodiment, the material 133 is positioned distal of the material 132. In that regard, the relative properties of the materials 132, 133 can be selected to provide a desired transition in stiffness along the length of the distal flexible element 122. For example, where the distal flexible element 122 extends from a rigid housing, it can be desirable to provide a gradual transition in stiffness from the housing to the distal flexible element 122. Accordingly, in some implementations the material 132 has an increased stiffness or durometer relative to the material 133 to facilitate a gradual transition in stiffness. The relative amounts of each materials 132, 133 utilized can be selected to achieve the desired stiffness transition along the length of the distal flexible element 122. Further, in some instances three or more materials having varying stiffness properties can be utilized in a similar manner.

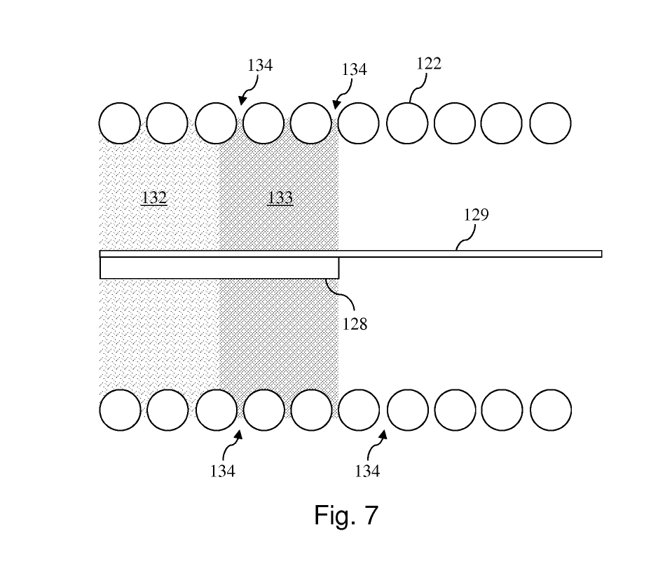

Referring now to FIG. 7, shown therein is another embodiment where multiple flexible materials are utilized within the distal flexible element 122, but where the distal core 128 is coupled to a shaping ribbon 129. In some implementations, the core member 128 is coupled to the shaping ribbon 129 utilizing a multi-flat transition as described in U.S. Patent Application No. 62/027,556, filed Jul. 22, 2014 (Attorney Docket No. 44755.1466/FM-0114), which is hereby incorporated by reference in its entirety. Further, in some instances the core member 126 extends through the proximal flexible element 120 and the housing 124 such that a distal section of the core member 126 defines the core member 128 within the distal flexible element 122. In some embodiments where the distal flexible element 122 has a length of approximately 3 cm, the core member 128 extends along the length of the distal flexible element 122 approximately 1 cm such that only the shaping ribbon 129 extends the last 2 cm of the distal flexible element 122.

In some instances, a method of forming or manufacturing a sensing guide wire in accordance with the present disclosure includes providing the requisite components and coupling them together in a manner to form the intravascular device 100. In that regard, the flexible element(s) can be filled, or partially filled, with the flexible adhesive(s) before and/or after coupling other components together. In that regard, the flexible adhesive(s) can be inserted into the distal flexible element using any suitable techniques, including wicking, injecting, flowing, and/or combinations thereof. In that regard, in some instances the flexible adhesive(s) have a starting viscosity in the range of 10 CPS to 80,000 CPS, with some implementations being between about 200 CPS and 60,000 CPS. In some instances, the flexible adhesive(s) are UV cured with a secondary heat or moisture cure due to ensure any hidden sections are cured. However, the adhesive(s) can be heat and/or moisture cure only adhesives in some instances.

Guide wires of the present disclosure can be connected to an instrument, such as a computing device (e.g. a laptop, desktop, or tablet computer) or a physiology monitor, that converts the signals received by the sensors into pressure and velocity readings. The instrument can further calculate Coronary Flow Reserve (CFR) and Fractional Flow Reserve (FFR) and provide the readings and calculations to a user via a user interface. In some embodiments, a user interacts with a visual interface to view images associated with the data obtained by the intravascular devices of the present disclosure. Input from a user (e.g., parameters or a selection) are received by a processor in an electronic device. The selection can be rendered into a visible display.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A sensing guide wire, comprising:
a flexible elongate member;
a sensing element coupled to a distal portion of the flexible elongate member; and
a flexible element extending distally from the sensing element, wherein the flexible element is at least partially filled along a longitudinal axis of the flexible element with one or more flexible adhesives.

2. The guide wire of claim 1, wherein the sensing element includes at least one of a pressure sensor and a flow sensor.

3. The guide wire of claim 1, further comprising a core element within the flexible element.

4. The guide wire of claim 3, wherein the core element is coupled to a shaping ribbon such that the core element extends only a portion of a total length of the flexible element.

5. The guide wire of claim 1, wherein the flexible element includes a coil.

6. The guide wire of claim 5, wherein the one or more flexible adhesives includes an adhesive selected from the group of adhesives consisting of urethane adhesives and silicone adhesives.

7. The guide wire of claim 1, wherein the one or more adhesives includes a first adhesive and a second adhesive, the second adhesive having an increased flexibility relative to the first adhesive.

8. The guide wire of claim 7, wherein the second adhesive is positioned distal of the first adhesive within a central lumen of the flexible element.

9. The guide wire of claim 8, wherein the flexible element includes a coil and wherein the flexible adhesive is spaced from the outer surface of the coil by a distance of at least ten percent of a diameter of a wire material forming the coil.

10. The guide wire of claim 9, wherein the coil has an outer diameter of approximately 0.014″, 0.018″, or 0.035″.

11. A method of forming a sensing guide wire, the method comprising:
coupling a sensing element to a distal portion of a flexible elongate member;
coupling a flexible element to the distal portion of the flexible elongate member such that the flexible element extends distally from the sensing element; and
at least partially filling the flexible element with one or more flexible adhesives.

12. The method of claim 11, wherein the sensing element includes at least one of a pressure sensor and a flow sensor.

13. The method of claim 11, wherein the step of filling the flexible element with a flexible adhesive is performed after coupling the flexible element to the distal portion of the flexible elongate member.

14. The method of claim 11, wherein the step of filling the flexible element with a flexible adhesive is performed before coupling the flexible element to the distal portion of the flexible elongate member.

15. The method of claim 11, wherein the flexible element includes a coil.

16. The method of claim 15, wherein the one or more flexible adhesives includes an adhesive selected from the group of adhesives consisting of urethane adhesives and silicone adhesives.

17. The method of claim 11, wherein at least partially filling the flexible element with the one or more adhesives includes introducing a first adhesive and a second adhesive into the flexible element, the second adhesive having an increased flexibility relative to the first adhesive.

18. The method of claim 17, wherein the second adhesive is introduced into the flexible element distal of the first adhesive.

19. The method of claim 18, wherein the first and second adhesives couple the flexible element to a distal core element.

20. The method of claim 19, wherein the distal core element is coupled to a shaping ribbon such that the distal core element extends only a portion of a total length of the flexible element.
